(19) United States
(12) Patent Application Publication
(10) Pub. No.: US 2011/0053969 A1
(43) Pub. Date: Mar. 3, 2011

(54) NOVEL DIAZASIPIROAKANES AND THEIR USE FOR TREATMENT OF CCR8 MEDIATED DISEASES

(75) Inventors: Stephen Connolly, Leicestershire (GB); Marco Skrinjar, Lund (SE)

(73) Assignee: ASTRAZENECA AB, Sodertalje (SE)

(21) Appl. No.: 11/910,580

(22) PCT Filed: Mar. 30, 2006
(86) PCT No.: PCT/SE2006/000390

§ 371 Date: Jun. 11, 2008

(30) Foreign Application Priority Data
Apr. 4, 2005 (SE) 0500741-4

(51) Int. Cl. A61K 31/438; C07D 221/20; A61P 11/06; A61P 11/00

(52) U.S. Cl. 514/278; 546/16

(57) ABSTRACT

The invention provides compounds of general formula. (II) wherein R and [symbol] are as defined in the specification, processes for their preparation, pharmaceutical compositions containing them and their use in therapy.

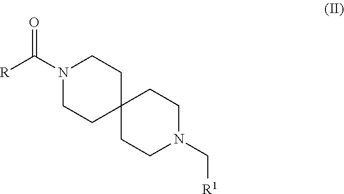

The present invention relates to novel diazaspiro compounds, processes for their preparation, pharmaceutical compositions containing them and their use in therapy.

Both the initial stages of a disease as well as the long-term tissue remodeling and muscle hypotrophy depend on recruitment of leukocytes to the inflammatory lesion. Leukocyte recruitment involves the migration of leukocytes into the diseased tissue from the blood vessel and their activation, which leads to progression of disease. The mechanism underlying this recruitment, chemotaxis, is similar both in classically defined immune mediated pathological conditions (i.e. allergic and autoimmune diseases) as well as others (i.e. atherosclerosis and Parkinson's disease). Thus, intervention of leukocyte recruitment to the inflamed target tissue constitutes an attractive novel therapeutic principle.

The chemokines are a large family (>50 members) of small 8- to 15-kDa secreted, is heparin-binding polypeptides with the primary function of controlling trafficking and activation of leukocytes. They are distinct from classical chemoattractants (i.e. bacterial derived N-formyl peptides, complement components, lipid molecules and platelet activating factor) on the basis of shared structural similarities. All chemokines have four conserved cysteines residues that form disulfide bonds, which are critical for the 3-D structure. The chemokines are further subclassed according to the position of the first two cysteines. The two major subclasses are the CC-chemokines, that have the cysteines adjacent, and the CXC-cytokines, that have the cysteines separated by one amino acid. The two other families, the C and the CX3C chemokines, are much smaller and only comprise one or a few members.

The specific biological effects of chemokines, including leukocyte recruitment, are mediated via interactions with a family of seven-transmembrane G-protein coupled receptors (GPCRs). The chemokine receptors are ˜350 amino acids in length and consist of a short extracellular N-terminus, seven transmembrane segments, and an intracellular C-terminus. The seven transmembrane domains are α-helical, and 3 intracellular and 3 extracellular loops exist between the domains.

So far 18 human chemokine receptors have been identified. Of these there are 11 CC chemokine receptors, 5 CXC receptors, 1 CX3C receptor and 1 C receptor. In general, CC chemokines are potent chemoattractants of monocytes and lymphocytes, but poor activators of neutrophils. Certain receptors bind multiple chemokines, for example, CCR1 binds CCL3, CCL5, CCL7 and CCL8, while other chemokine receptors have a more restricted binding profile. This ligand specificity, together with chemokine receptor expression patterns on particular leukocyte subsets, accounts for the regulated, restricted, and specific trafficking of cells into inflammatory lesions. Chemotaxis of inflammatory cells towards a chemokine gradient is initiated by signals mediated by the intracytoplasmatic tail of the chemokine receptor. The downstream signals involve the PI3Kγ, the MAPK and the PKC pathways, among others.

The accumulation of immune cells at a site of allergic inflammation occurs within 6-48 hours after allergen challenge and is a hallmark of allergic diseases. Studies have shown that antigen-specific CD4⁺ T cells are detected in lung tissue in asthmatic patients after exposure to the allergen. Although infiltrating T cells are relatively few in number compared to eosinophils, compelling evidence has demonstrated essential roles for T cells in orchestrating the inflammatory process in human asthma. A close correlation exists in humans between the level of TH2 cytokines produced by T cells, serum level of IgE and prevalence of asthma.

The human CCR8 receptor has been shown to interact with the human chemokine CCL1 (I-309). This chemokine is a potent eosinophil, T cell and endothelial cell chemoattractant. The receptor has been shown to be transiently upregulated on polarized TH2 cells after optimal TCR cross linkage in presence of costimulatory signals (i.e. CD28). The coordinated upregulation of CCR8 on activated T cells after antigen challenge indicates that it contributes to redistribution of the activated T cells to the inflammatory foci within the inflamed tissue expressing CCL1. Indeed, in vivo models of allergic airway inflammation using mice deficient in CCR8 expression have shown a profound block in recruitment of effector T cells to the inflamed lung tissue and production of TH2 cytokines. Moreover, T cells infiltrating the human airway subepithelium during allergen challenge have been shown to be CCR8 positive. Importantly, the number of CCR8 positive cells migrating into the airway submucosa following allergen challenge has been shown to correlate with decreases in FEV1.

Considering the significant role CCR8 plays in TH2 cell chemotaxis, and the importance of TH2 cells in allergic conditions such as asthma, CCR8 represents a good target for drug development in treatment of respiratory diseases, including asthma, chromic obstructive pulmonary disease and rhinitis.

International patent application number WO2005/040167 describes diazaspiro compounds having activity at the CCR8 receptor.

A desirable property for a drug acting at the CCR8 receptor is that it has high potency e.g. as determined by its ability to inhibit the activity of the CCR8 receptor. It is also desirable for such drugs to possess good metabolic stability in order to enhance drug efficacy. Stability against human microsomal metabolism in vitro is indicative of stability towards metabolism in vivo.

The present inventors have identified a set of compounds which show a surprising combination of high potency against CCR8 (determined from inhibition of CCL1 binding to CCR8) and good stability against human microsomal metabolism in vitro.

In accordance with the present invention, there is provided a compound of formula:

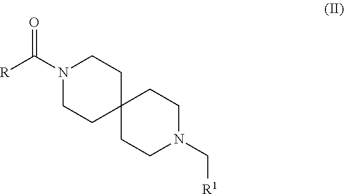

wherein R is pyridin-2-one or N—C₁-C₄ alkyl pyridin-2-one,
or wherein R is pyridin-2-one or N—C₁-C₄ alkyl pyridin-2-one each of which is substituted by a group selected from CF₃, halogen or C₁-C₄ alkyl;
R¹ represents the group:

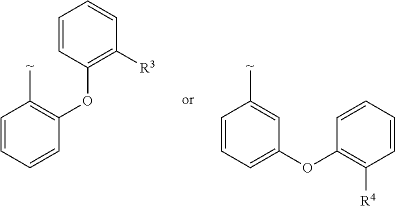

and R³ and R⁴ are independently ethoxy or methoxy;
or a pharmaceutically acceptable salt thereof.

Without being bound to any particular theory, it is believed that the pyridin-2-one may contribute towards enhancing metabolic stability and the alkoxy-substituted phenoxy group on the right hand side of the molecule may contribute towards enhancing CCR8 potency.

The teen alkyl, whether alone or as part of another group, includes straight chain and branched chain alkyl groups.

The term “pyridin-2-one” refers to the following structure:

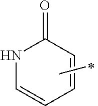

wherein the point of attachment (*) to the remainder of formula II can be made from any of the remaining carbon atoms (other than the carbonyl carbon).

The term “N—C₁-C₄ alkyl pyridin-2-one” refers to a pyridin-2-one group as defined above which has been substituted on its nitrogen by a C₁-C₄ alkyl group (e.g., methyl, ethyl, propyl). Preferably, the N—C₁-C₄ alkyl pyridin-2-one is N-methylpyridin-2-one.

R may, for example, be one of the following:

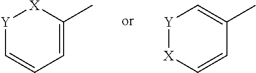

where X is C═O and Y is NH or N substituted by C₁-C₄ alkyl, and wherein R may be either unsubstituted (other than the aforementioned N-substitution) or have a substituent which is CF₃, halogen or C₁-C₄ alkyl.

In an embodiment of the present invention, R is substituted by a group independently selected from CF₃, halogen (e.g. chlorine, fluorine or bromine, preferably chlorine) or C₁-C₄ alkyl (e.g. methyl). In this regard, the substituent group is connected to the pyridin-2-one (or N—C₁-C₄ alkyl pyridine-2-one) on a ring-carbon other than the carbonyl carbon. For example, R may be substituted as shown below:

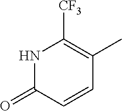

When R contains a substituent (other than being alkylated on its nitrogen atom), R preferably contains a single substituent selected from CF₃, halogen or C₁-C₄ alkyl.

In another embodiment of the present invention, R is not substituted.

R³ and R⁴ can be ethoxy or methoxy. In an embodiment of the present invention, R³ and R⁴ are methoxy. In this regard, compounds with a particularly advantageous combination of high potency and good stability against human microsomal metabolism in vitro were found to be those with R³ and R⁴ being methoxy.

In an embodiment of the present invention, R is pyridin-2-one or N—C₁-C₄ alkyl pyridin-2-one; or R is pyridin-2-one or N—C₁-C₄ alkyl pyridin-2-one each of which is substituted by a group selected from CF₃, halogen or C₁-C₄ alkyl; R¹ represents the group:

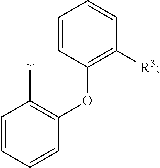

and R³ is methoxy or ethoxy.

In another embodiment of the present, R is pyridin-2-one or N—C₁-C₄ alkyl pyridin-2-one; or R is pyridin-2-one or N—C₁-C₄ alkyl pyridin-2-one each of which is substituted by a group selected from CF₃, halogen or C₁-C₄ alkyl; R¹ represents the group:

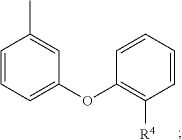

and R⁴ is methoxy or ethoxy.

In a further embodiment of the present invention, R is pyridin-2-one or N—C₁-C₄ alkyl pyridin-2-one; or R is pyridin-2-one or N—C₁-C₄ alkyl pyridin-2-one each of which is substituted by a CF₃ group; R¹ represents the group:

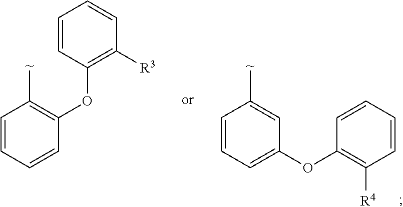

and R³ and R⁴ is methoxy.

For compounds of formula (II) which are capable of existing in stereoisomeric forms, it will be understood that the invention encompasses all geometric and optical isomers of the compounds and mixtures thereof including racemates. Tautomers and mixtures thereof also form an aspect of the present invention. Accordingly, the presently claimed pyrid-2-ones encompass the corresponding hydroxyl pyridine tautomers.

Preferred compounds of the present invention include:
3-({9-[2-(2-methoxyphenoxy)benzyl]-3,9-diazaspiro[5.5]undec-3-yl}carbonyl)-1-methylpyridin-2(1H)-one,
3-({9-[2-(2-methoxyphenoxy)benzyl]-3,9-diazaspiro[5.5]undec-3-yl}carbonyl)pyridin-2(1H)-one,
5-({9-[2-(2-methoxyphenoxy)benzyl]-3,9-diazaspiro[5.5]undec-3-yl}carbonyl)-6-(trifluoromethyl)pyridin-2(1H-one,
3-({9-[3-(2-methoxyphenoxy)benzyl]-3,9-diazaspiro[5.5]undec-3-yl}carbonyl)pyridin-2(1H)-one,
   or a pharmaceutically acceptable salt thereof.

According to the present invention there is also provided a process for the preparation of compounds of formula (II) and salts thereof which comprises

(a) reacting a compound of formula (III):

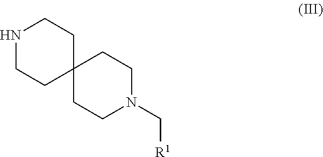

where R¹ is as defined in formula (II), with a compound of formula (IV):

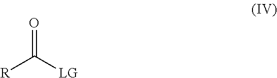

where R is as defined in formula (II) and LG is a suitable leaving group, or
(b) reaction of a compound of formula (V)

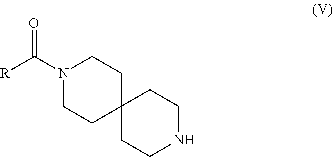

wherein R is as defined in formula (II), with an aldehyde compound of formula (VI):

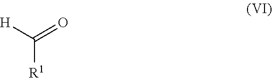

wherein R¹ is as defined in formula (II), or
(c) reaction of a compound of formula (V) defined above with a compound of formula (VII)

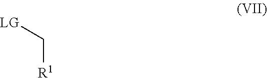

wherein R¹ is as defined in formula (II) and LG is a leaving group.

A compound of formula (III) can be prepared by process (d) by reacting a compound of formula (VIII)

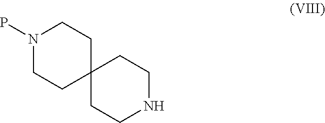

in which P is a protecting group, with a compound of formula (VI) as defined above, and subsequently removing the protecting group P.

A compound of formula (III) can also be prepared by process (e) by reacting a compound of formula (VIII) with a compound of formula (VII), and subsequently removing the protecting group P.

A compound of formula (V) can be prepared by process (f) by reacting a compound of formula (IX):

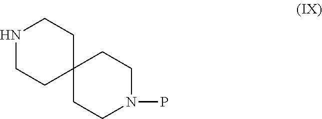

where P is a suitable protecting group with a compound of formula (IV) as defined above, and subsequently removing the protecting group P.

Process (a) may be carried out using standard coupling reactions that are well know in the art. A suitable leaving group LG is, for example OH or chlorine, preferably OH. The coupling reaction may typically carried out using activating reagents such as N-[(1H-1,2,3-benzotriazol-1-yloxy)(dimethylamino)methylene]-N-methylmethanaminium hexafluorophosphate (HBTU), N-[(dimethylamino)(3H-[1,2,3]triazolo[4,5-b]pyridin-3-yloxy)methylene]-N-methylmethanaminium hexafluorophosphate (HATU), or (benzotriazol-1-yloxy)tripyrrolidinophosphonium hexafluorophosphate (PYBOP). Typically, the reaction is carried out in the presence of a suitable base (e.g. triethylamine) and an organic solvent (e.g. dichloromethane) at a suitable temperature (e.g. room temperature).

Process (b) may be carried out using standard reductive amination procedures which are well known in the art. Typically, the reaction is carried out in the presence of sodium triacetoxyborohydride [NaBH(OAc)₃]. Typically, the reaction is carried out in the presence of a suitable base (e.g. triethylamine) and an organic solvent (e.g. dichloromethane) at a suitable temperature (e.g. room temperature).

Process (c) may be carried out in a suitable organic solvent (e.g. DMF) at a suitable temperature (e.g. room temperature). The use of leaving groups are well known in the art for this type of reaction. Examples of typical leaving groups are halo, alkoxy, trifluoromethanesulfonyloxy, methanesulfonyloxy, or p-toluenesulfonyloxy. Typically, the leaving group is a halogen such as chlorine or bromine.

The coupling step of process (d) may be carried out according to the conditions described for process (b) above. The coupling step of process (e) may be carried out according to the conditions described for process (c) above. The coupling step of process (f) may be carried out according to the conditions described for process (a) above. An example of a typical protecting group P used in processes (d), (e) and (f) is tert-butyloxycarbonyl (t-boc). However, other suitable protecting groups may be used. In this regard, the protection and deprotection of functional groups is fully described in ‘Protective Groups in Organic Chemistry’, edited by J. W. F. McOmie, Plenum Press (1973), and ‘Protective Groups in Organic Synthesis’, 2nd edition, T. W. Greene & P. G. M. Wuts, Wiley-Interscience (1991). After the coupling the protecting group P can be removed.

Compounds of formulae (IV), (VI), (VII), (VIII), and (IX) are either commercially available, are well known in the literature or may be prepared easily using known techniques, for example as shown in the accompanying Examples. U.S. Pat. No. 5,451,578 (Claremon et al.) describes, under example 1 of the patent, a process for synthesising tert-butyl 3,9-diazaspiro[5.5]undecane-3-carboxylate (corresponding to compound (IX) with P as tert-butyloxycarbonyl).

In so far as the intermediates referred to in the processes of the present invention are capable of forming salts, the processes of the invention described above encompass the use of the intermediates in salt faun or free form.

The compounds of formula (II) above may be converted to a pharmaceutically acceptable salt thereof, preferably an acid addition salt such as a hydrochloride, hydrobromide, phosphate, acetate, fumarate, maleate, tartrate, citrate, oxalate, methanesulphonate or p-toluenesulphonate.

The compounds of formula (II) and pharmaceutically acceptable salts thereof may exist in solvated, for example hydrated, as well as unsolvated forms, and the present invention encompasses all such solvated forms.

The compounds of formula (II) have activity as pharmaceuticals, in particular as modulators of chemokine receptor (especially CCR8) activity, and may be used in the treatment (therapeutic or prophylactic) of conditions/diseases in human and non-human animals which are exacerbated or caused by excessive or dysregulated production of chemokines. Examples of such conditions/diseases include:
(1) (the respiratory tract) obstructive airways diseases including chronic obstructive pulmonary disease (COPD), asthma, such as bronchial, allergic, intrinsic, extrinsic and dust asthma, particularly chronic or inveterate asthma (e.g. late asthma and airways hyper-responsiveness), bronchitis, acute, allergic, atrophic rhinitis and chronic rhinitis including rhinitis caseosa, hypertrophic rhinitis, rhinitis purulenta, rhinitis sicca and rhinitis medicamentosa, membranous rhinitis including croupous, fibrinous and pseudomembranous rhinitis and scrofoulous rhinitis, seasonal rhinitis including rhinitis nervosa (hay fever) and vasomotor rhinitis, sarcoidosis, farmer's lung and related diseases, fibroid lung and idiopathic interstitial pneumonia,
(2) (bone and joints) gout, rheumatoid arthritis, seronegative spondyloarthropathies (including ankylosing spondylitis, psoriatic arthritis and Reiter's disease), Behcet's disease, Sjogren's syndrome and systemic sclerosis,
(3) (skin) pruritis, scleroderma, otitus, psoriasis, atopical dermatitis, contact dermatitis and other eczematous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia greata and vernal conjunctivitis, lupus,
(4) (gastrointestinal tract) Coeliac disease, proctitis, eosinopilic gastro-enteritis, mastocytosis, inflammatory bowel diseases such as Crohn's disease, ulcerative colitis, ileitis and enteritis, food-related allergies which have effects remote from the gut, e.g., migraine, rhinitis and eczema,
(5) (central and peripheral nervous system) Neurodegenerative diseases and dementia disorders, e.g. Alzheimer's disease, amyotrophic lateral sclerosis and other motor neuron diseases, Creutzfeldt-Jacob's disease and other prion diseases, HIV encephalopathy (AIDS dementia complex), Huntington's disease, frontotemporal dementia, Lewy body dementia and vascular dementia, polyneuropathies, e.g. Guillain-Barré syndrome, chronic inflammatory demyelinating polyradiculoneuropathy, multifocal motor neuropathy, plexopathies, CNS demyelination, e.g. multiple sclerosis, acute disseminated/haemorrhagic encephalomyelitis, and subacute sclerosing panencephalitis, neuromuscular disorders, e.g. myasthenia gravis and Lambert-Eaton syndrome, spinal disorders, e.g. tropical spastic paraparesis, and stiff-man syndrome: paraneoplastic syndromes, e.g. cerebellar degeneration and encephalomyelitis, CNS trauma, migraine, stroke and correctum diseases such as meningitis
(6) (other tissues and systemic disease) hepatitis, vasculitis, spondyloarthropathies, vaginitis, glomerulonephritis, myositis, atherosclerosis, Acquired Immunodeficiency Syndrome (AIDS), lupus erythematosus, systemic lupus, erythematosus, Hashimoto's thyroiditis, type I diabetes, nephrotic syndrome, eosinophilia fascitis, hyper IgE syndrome, lepromatous leprosy, and idiopathic thrombocytopenia pupura, post-operative adhesions, and sepsis.
(7) (allograft and xenograft rejection) acute and chronic following, for example, transplantation of kidney, heart, liver, lung, bone marrow, skin and cornea, and chronic graft versus host disease,
(8) Cancer, carcinoma & tumour metastasis, including that of the bladder, breast, colon, kidney, liver, lung, ovary, pancreas, stomach, cervix, thyroid and skin, especially non-small cell lung cancer (NSCLC), malignant melanoma, prostate cancer and squamous sarcoma. Hematopoietic tumors of lymphoid lineage, including acute lymphocytic leukemia, B cell lymphoma and Burketts lymphoma, Hodgkins Lymphoma, Acute Lymphoblastic Leukemia. Hematopoietic tumors of myeloid lineage, including acute and chronic myelogenous leukemias and promyelocytic leukemia. Tumors of mesenchymal origin, including fibrosarcoma and rhabdomyosarcoma, and other tumors, including melanoma, seminoma, tetratocarcinoma, neuroblastoma and glioma.
(9) All diseases that result from a general inbalance of the immune system and resulting in increased atopic inflammatory reactions.
(10) Cystic fibrosis, re-perfusion injury in the heart, brain, peripheral limbs and other organs.
(11) Burn wounds & chronic skin ulcers
(12) Reproductive Diseases (e.g. Disorders of ovulation, menstruation and implantation, Pre-term labour, Endometriosis)
(13) thrombosis
(14) infectious diseases such as HIV infection and other viral infections, bacterial infections.

Thus, the present invention provides a compound of formula (II) or a pharmaceutically-acceptable salt thereof, as hereinbefore defined for use in therapy.

In a still further aspect, the present invention provides the use of a compound of formula (II) or a pharmaceutically acceptable salt thereof, as hereinbefore defined in the manufacture of a medicament for the treatment of human diseases or conditions in which modulation of chemokine receptor activity, particularly CCR8 activity, is beneficial.

In the context of the present specification, the term “therapy” also includes “prophylaxis” unless there are specific indications to the contrary. The terms “therapeutic” and “therapeutically” should be construed accordingly.

The invention still further provides a method of treating a chemokine mediated disease wherein the chemokine binds to a chemokine (especially CCR8) receptor, which comprises administering to a patient a therapeutically effective amount of a compound of formula (II) or a pharmaceutically acceptable salt thereof.

The invention also provides a method of treating a respiratory disease, such as asthma, COPD or rhinitis, in a patient suffering from, or at risk of, said disease, which comprises administering to the patient a therapeutically effective amount of a compound of formula (II) or a pharmaceutically acceptable salt thereof, as hereinbefore defined.

For the above-mentioned therapeutic uses the dosage administered will, of course, vary with the compound employed, the mode of administration, the treatment desired and the disorder indicated.

The compounds of formula (II) and pharmaceutically acceptable salts thereof may be used on their own but will generally be administered in the fowl of a pharmaceutical composition in which the compound/salt/solvate (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (II) or a pharmaceutically acceptable salt thereof, as hereinbefore defined, in association with a pharmaceutically acceptable adjuvant, diluent or carrier.

The invention further provides a process for the preparation of a pharmaceutical composition of the invention which comprises mixing a compound of formula (II) or a pharmaceutically acceptable salt, as hereinbefore defined, with a pharmaceutically acceptable adjuvant, diluent or carrier.

The pharmaceutical compositions may be administered topically (e.g. to the lung and/or airways or to the skin) in the form of solutions, suspensions, heptafluoroalkane aerosols and dry powder formulations, or systemically, e.g. by oral administration in the form of tablets, capsules, syrups, powders or granules, or by parenteral administration in the form of solutions or suspensions, or by subcutaneous administration or by rectal administration in the form of suppositories or transdermally. Preferably the compound of the invention is administered orally.

The invention further relates to combination therapies wherein a compound of the invention or a pharmaceutically acceptable salt thereof, or a pharmaceutical composition or formulation comprising a compound of formula (II) is administered concurrently or sequentially with therapy and/or an agent for the treatment of any one of asthma, allergic rhinitis, cancer, COPD, rheumatoid arthritis, psoriasis, inflammatory bowel diseases, osteoarthritis or osteoporosis.

In particular, for the treatment of the inflammatory diseases rheumatoid arthritis, psoriasis, inflammatory bowel disease, COPD, asthma and allergic rhinitis the compounds of the invention may be combined with agents such as TNF-α inhibitors such as anti-TNF monoclonal antibodies (such as Remicade, CDP-870 and D₂E₇ and TNF receptor immunoglobulin molecules (such as Enbrel®), non-selective COX-1/COX-2 inhibitors (such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin), COX-2 inhibitors (such as meloxicam, celecoxib, rofecoxib, valdecoxib and etoricoxib) low dose methotrexate, lefunomide, ciclesonide, hydroxychloroquine, d-penicillamine, auranofin or parenteral or oral gold.

The present invention still further relates to the combination of a compound of the invention together with a leukotriene biosynthesis inhibitor, 5-lipoxygenase (5-LO) inhibitor or 5-lipoxygenase activating protein (FLAP) antagonist such as zileuton, ABT-761, fenleuton, tepoxalin, Abbott-79175, Abbott-85761, N-(5-substituted)-thiophene-2-alkylsulfonamides, 2,6-di-tent-butylphenol hydrazones, methoxytetrahydropyrans such as Zeneca ZD-2138, the compound SB-210661, pyridinyl-substituted 2-cyanonaphthalene compounds such as L-739,010,2-cyanoquinoline compounds such as L-746,530, indole and quinoline compounds such as MK-591, MK-886, and BAY x 1005.

The present invention still further relates to the combination of a compound of the invention together with a receptor antagonist for leukotrienes LTB₄, LTC₄, LTD₄, and LTE₄ selected from the group consisting of the phenothiazin-3-ones such as L-651,392, amidino compounds such as CGS-25019c, benzoxalamines such as ontazolast, benzenecarboximidamides such as BIIL 284/260, and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention together with a PDE4 inhibitor including inhibitors of the isoform PDE4D.

The present invention still further relates to the combination of a compound of the invention together with a antihistaminic H₂ receptor antagonists such as cetirizine, loratadine, desloratadine, fexofenadine, astemizole, azelastine, and chlorpheniramine.

The present invention still further relates to the combination of a compound of the invention together with a gastroprotective H₂ receptor antagonist.

The present invention still further relates to the combination of a compound of the invention together with an α₁.- and α₂.-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride.

The present invention still further relates to the combination of a compound of the invention together with anticholinergic agents such as ipratropium bromide, tiotropium bromide, oxitropium bromide, pirenzepine, and telenzepine.

The present invention still further relates to the combination of a compound of the invention together with a β₁- to β₄-adrenoceptor agonists such as metaproterenol, isoproterenol, isoprenaline, albuterol, salbutamol, formoterol, salmeterol, terbutaline, orciprenaline, bitolterol mesylate, and pirbuterol, or methylxanthanines including theophylline and aminophylline, sodium cromoglycate, or muscarinic receptor (M1, M2, and M3) antagonist.

The present invention still further relates to the combination of a compound of the invention together with an insulin-like growth factor type I (IGF-1) mimetic.

The present invention still further relates to the combination of a compound of the invention together with an inhaled glucocorticoid with reduced systemic side effects, such as prednisone, prednisolone, flunisolide, triamcinolone acetonide, beclomethasone dipropionate, budesonide, fluticasone propionate, and mometasone furoate.

The present invention still further relates to the combination of a compound of the invention together with an inhibitor of matrix metalloproteases (MMPs), i.e., the stromelysins, the collagenases, and the gelatinases, as well as aggrecanase, especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-12.

The present invention still further relates to the combination of a compound of the invention together with other modulators of chemokine receptor function such as CCR1, CCR2, CCR2A, CCR2B, CCR3, CCR4, CCR5, CCR6, CCR7, CCR8, CCR9, CCR10 and CCR11 (for the C-C family), CXCR1, CXCR2, CXCR3, CXCR4 and CXCR5 (for the C-X-C family) and CX₃CR1 for the C-X₃-C family.

The present invention still further relates to the combination of a compound of the invention together with antiviral agents such as Viracept, AZT, aciclovir and famciclovir, and antisepsis compounds such as Valant.

The present invention still further relates to the combination of a compound of the invention together with cardiovascular agents such as calcium channel blockers, lipid lowering agents such as statins, fibrates, beta-blockers, Ace inhibitors, Angiotensin-2 receptor antagonists and platelet aggregation inhibitors.

The present invention still further relates to the combination of a compound of the invention together with CNS agents such as antidepressants (such as sertraline), anti-Parkinsonian drugs (such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti-Alzheimer's drugs such as donepezil, tacrine, COX-2 inhibitors, propentofylline or metrifonate.

The present invention still further relates to the combination of a compound of the invention together with (i) tryptase inhibitors, (ii) platelet activating factor (PAF) antagonists, (iii) interleukin converting enzyme (ICE) inhibitors, (iv) IMPDH inhibitors, (v) adhesion molecule inhibitors including VLA-4 antagonists, (vi) cathepsins, (vii) MAP kinase inhibitors, (viii) glucose-6 phosphate dehydrogenase inhibitors, (ix) kinin-B₁- and B₂-receptor antagonists, (x) anti-gout agents, e.g., colchicine, (xi) xanthine oxidase inhibitors, e.g., allopurinol, (xii) uricosuric agents, e.g., probenecid, sulfinpyrazone, and benzbromarone, (xiii) growth hormone secretagogues, (xiv) transforming growth factor (TGFβ), (xv) platelet-derived growth factor (PDGF), (xvi) fibroblast growth factor, e.g., basic fibroblast growth factor (bFGF), (xvii) granulocyte macrophage colony stimulating factor (GM-CSF), (xviii) capsaicin cream, (xix) Tachykinin NK₁ and NK₃ receptor antagonists selected from the group consisting of NKP-608C, SB-233412 (talnetant), and D-4418, (xx) elastase inhibitors selected from the group consisting of UT-77 and ZD-0892, (xxi) TNFα converting enzyme inhibitors (TACE), (xxii) induced nitric oxide synthase inhibitors (iNOS) or (xxiii) chemoattractant receptor-homologous molecule expressed on TH2 cells, (CRTH2 antagonists).

The compounds of the present invention may also be used in combination with osteoporosis agents such as roloxifene, droloxifene, lasofoxifene or fosomax and immunosuppressant agents such as FK-506, rapamycin, cyclosporine, azathioprine, and methotrexate.

The compounds of the invention may also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, rofecoxib and etoricoxib, analgesics and intraarticular therapies such as corticosteroids and hyaluronic acids such as hyalgan and synvisc and P2X7 receptor antagonists.

The compounds of the invention can also be used in combination with existing therapeutic is agents for the treatment of cancer. Suitable agents to be used in combination include:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas), antimetabolites (for example antifolates such as fluoropyrimidines like 5-fluorouracil and tegafur, raltitrexed, methotrexate, cytosine arabinoside, hydroxyurea, gemcitabine and paclitaxel (Taxol®), antitumour antibiotics (for example anthracyclines like adriamycin, bleomycin, doxorubicin, daunomycin, epirubicin, idarubicin, mitomycin-C, dactinomycin and mithramycin), antimitotic agents (for example vinca alkaloids like vincristine, vinblastine, vindesine and vinorelbine and taxoids like taxol and taxotere), and topoisomerase inhibitors (for example epipodophyllotoxins like etoposide and teniposide, amsacrine, topotecan and camptothecin),
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride,
(iii) Agents which inhibit cancer cell invasion (for example metalloproteinase inhibitors like marimastat and inhibitors of urokinase plasminogen activator receptor function),
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family,
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin),
(vi) vascular damaging agents such as Combretastatin A4 and compounds disclosed in International Patent Applications WO 99/02166, WO00/40529, WO 00/41669, WO01/92224, WO02/04434 and WO02/08213,
(vii) antisense therapies, for example those which are directed to the targets listed above, such as ISIS 2503, an anti-ras antisense,
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy, and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies.

The invention will now be further explained by reference to the following illustrative examples.

### General Procedures

### HPLC Conditions

a. Method A

HPLC method A was performed with Agilent 1100 series machines on Kromassil© C18 5 μm 3.0×100 mm column. Aqueous phase was water/TFA (99.8/0.1) and organic phase was acetonitrile/TFA (99.92/0.08). Flow was 1 mL/min and gradient was set from 10 to 100% of organic phase during 20 minutes. Detection was carried out on 220, 254 and 280 nm.

b. Method B

HPLC method B was performed with Agilent 1100 series machines on XTerra® RP₈ 5 μm 3.0×100 mm column. Aqueous phase was 15 nM NH3 in water and organic phase was acetonitrile. Flow was 1 mL/min and gradient was set from 10 to 100% of organic phase during 20 minutes. Detection was carried out on 220, 254 and 280 nm.

### Starting materials for Examples 1 to 4

### Intermediate C

### 3-[2-(2-methoxyphenoxy)benzyl]-3,9-diazaspiro[5.5]undecane dihydrochloride

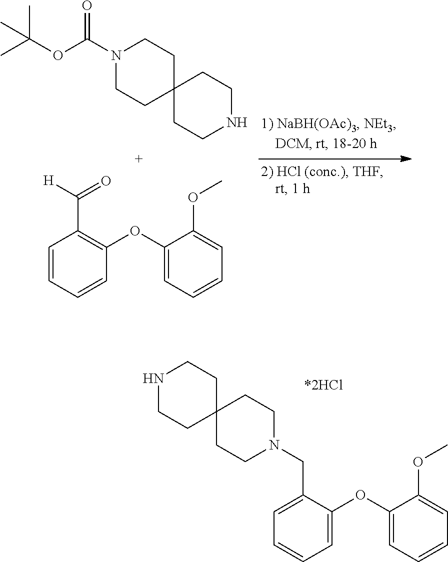

### a) tert-butyl 9-[2-(2-methoxyphenoxy)benzyl]-3,9-diazaspiro[5.5]undecane-3-carboxylate

tert-Butyl 3,9-diazaspiro[5.5]undecane-3-carboxylate hydrochloride (1.5 g, 5.2 mmol), 2-(2-methoxyphenoxy)benzaldehyde (1.24 g, 5.4 mmol), triethylamine (1.08 mL, 7.74 mmol) and sodium triacetoxyborohydride (1.23 g, 5.8 mmol) was dissolved in dichloromethane (40 mL) and dry DMF (15 mL). The pH was adjusted to 4 with AcOH and the mixture was stirred at room temperature over night. Another batch of sodium triacetoxyborohydride (1.0 g, 4.72 mmol) was added and the mixture was stirred at 40° C. for 2 hrs. The mixture was diluted with EtOAc (150 mL) and washed with sodium bicarbonate-solution, H₂O and brine and dried over Na₂SO₄ and evaporated. The crude product was purified using column chromatography on SiO₂ eluting with Heptane:EtOAc 4:1+2 vol % NEt₃ affording 1.27 g (53%) of the title compound as a colourless oil.

¹H NMR (400 MHz, DMSO-D6) δ 7.42 (dd, J=7.5, 1.5 Hz, 1H), 7.17-7.10 (m, 3H), 7.04 (td, J=7.4, 0.9 Hz, 1H), 6.95-6.88 (m, 2H), 6.84 (d, J=7.6 Hz, 1H), 6.58 (d, J=8.0 Hz, 1H), 3.74 (s, 3H), 3.54 (s, 2H), 3.30-3.23 (m, 6H), 2.40-2.34 (m, 4H), 1.46-1.40 (m, 12H), 1.38 (s, 11H), 1.34-1.29 (m, 14H)

APCI-MS m/z: 467.3 [MH+]

### b) 3-[2-(2-methoxyphenoxy)benzyl]-3,9-diazaspiro[5.5]undecane dihydrochloride

The oil from part a) was dissolved in THF (100 mL) and conc. HCl (20 mL) was added and the mixture was stirred at room temperature for 1 hr. The solvents were evaporated and the crude product was evaporated twice with toluene and ethanol to remove traces of water, affording 1.59 g (quant.) of the title compound as a slightly purple oil. Some toluene (12 wt %) was still left in the compound, which did not disappear even after 24 hrs under vacuum.

¹H NMR (400 MHz, CD3OD) δ 7.57 (dd, J=7.6, 1.6 Hz, 1H), 7.38-7.27 (m, 2H), 7.24-7.08 (m, 7H (+toluene)), 7.05 (td, J=7.7, 1.4 Hz, 1H), 6.60 (d, J=8.3 Hz, 1H), 4.55 (s, 2H), 3.75 (s, 3H), 3.64-3.49 (m, 4H), 3.25-3.19 (m, 4H), 2.32 (s, 2H (toluene)), 2.06 (d, J=14.7 Hz, 2H), 1.95 (t, J=5.9 Hz, 2H), 1.89-1.62 (m, 6H)

APCI-MS m/z: 367.5 [MH+]

### Intermediate D

### 3-[3-(2-methoxyphenoxy)benzyl]-3,9-diazaspiro[5.5]undecane dihydrochloride

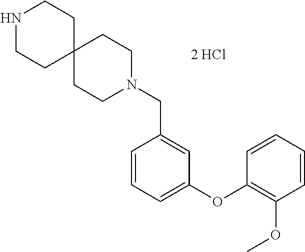

### a) 3-(2-methoxyphenoxy)benzaldehyde

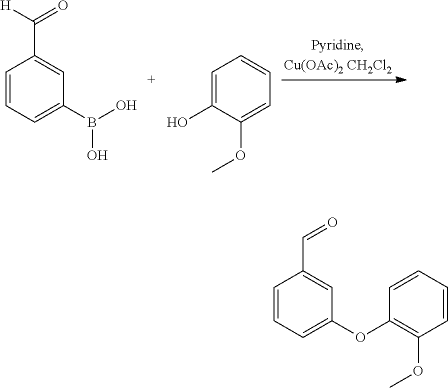

(3-Formylphenyl)boronic acid (5.0 g, 33 mmol) and guaiacol (2.8 g, 22 mmol) were mixed with Cu(OAc)₂ (4.0 g, 22 mmol), 4 Å molecular sieves and pyridine (9 mL) in dry dichloromethane (150 mL) and the resulting mixture was stirred overnight at room temperature. The reaction mixture was filtered and concentrated. Column chromatography on SiO₂ gave the title compound as an oil (1.7 g, 23%).

¹H NMR (400 MHz, CDCl3) δ 9.95 (s, 1H), 7.58-7.54 (m, 1H), 7.47 (t, J=7.8 Hz, 1H), 7.38-7.34 (m, 1H), 7.26-7.19 (m, 2H), 7.08-7.02 (m, 2H), 7.01-6.95 (m, 1H), 3.82 (s, 3H)

GC-MS m/z: 228.0 [M]

### b) tert-butyl 9-[3-(2-methoxyphenoxy)benzyl]-3,9-diazaspiro[5.5]undecane-3-carboxylate

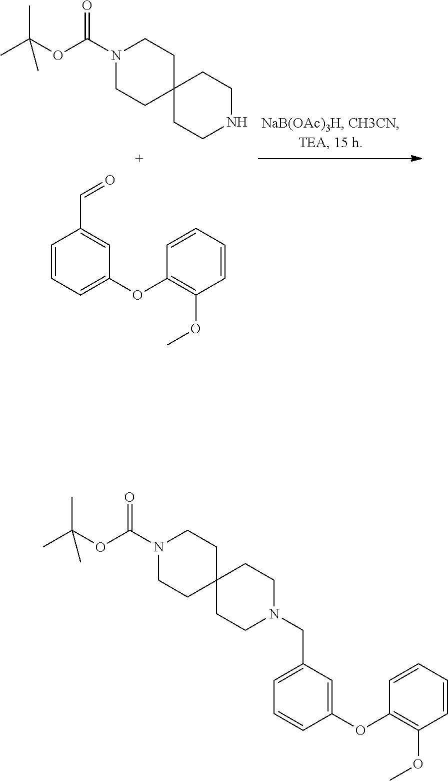

A mixture of tert-butyl 3,9-diazaspiro[5.5]undecane-3-carboxylate hydrochloride (1.4 g, 5.0 mmol), 3-(2-methoxyphenoxy)benzaldehyde (1.7 g, 7.5 mmol, triethylamine (1 mL, 7.5 mmol), sodium triacetoxyborohydride (1.6 g, 7.5 mmol) and acetonitrile were heated at reflux overnight. The reaction mixture was partitioned between ethyl acetate and saturated sodium hydrogen carbonate solution. The organic layer was isolated and evaporated to dryness. Column chromatography on SiO₂ gave the title compound (1.5 g, 64%).

¹H NMR (400 MHz, DMSO-D6) δ 7.26-7.14 (m, 3H), 7.04-6.90 (m, 3H), 6.76 (s, 1H), 6.71-6.66 (m, 1H), 3.39 (s, 2H), 3.31 (s, 5H), 3.29-3.23 (m, 4H), 2.33-2.25 (m, 4H), 1.43-1.36 (m, 11H), 1.35-1.27 (m, 4H)

APCI-MS m/z: 467.3 [MH+]

### c) 3-[3-(2-methoxyphenoxy)benzyl]-3,9-diazaspiro[5.5]undecane dihydrochloride

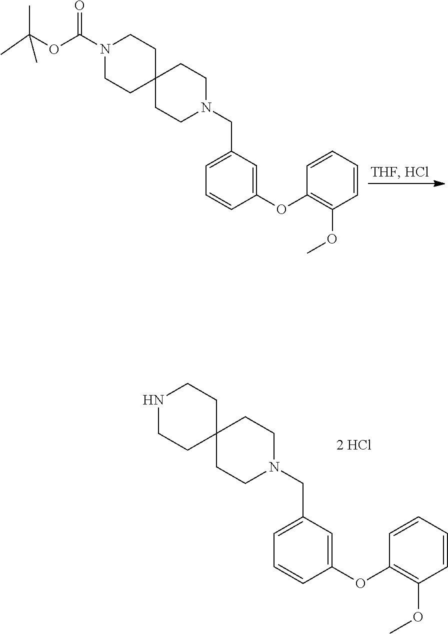

To a solution of tert-butyl 9-[3-(2-methoxyphenoxy)benzyl]-3,9-diazaspiro[5.5]undecane-3-carboxylate (1.6 g, 3.4 mmol) in 50 mL of THF was added 7 mL of conc. HCl. After 2 h to stirring at room temperature the reaction mixture was evaporated and co-evaporated three times with methanol and toluene. The title compound was obtained as a white solid.

¹H NMR (400 MHz, DMSO-D6) δ 7.37 (t, J=7.9 Hz, 1H), 7.29 (d, J=7.7 Hz, 1H), 7.26-7.16 (m, 2H), 7.14 (s, 1H), 7.10-7.05 (m, 1H), 7.02-6.96 (m, 1H), 6.88-6.81 (m, 1H), 4.25 (d, J=5.4 Hz, 2H), 3.73 (s, 3H), 3.13-2.94 (m, 8H), 1.88-1.64 (m, 6H), 1.56-1.47 (m, 2H)

APCI-MS m/z: 367.2 [MH+]

### EXAMPLE 1

### 3-({9-[2-(2-methoxyphenoxy)benzyl]-3,9-diazaspiro[5.5]undec-3-yl}carbonyl)-1-methylpyridin-2(1H)-one

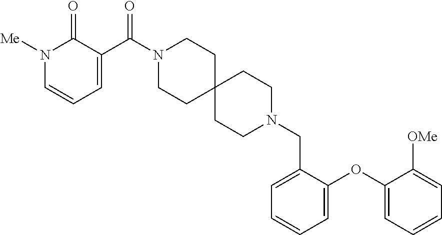

### a) N-methyl-2-hydroxynicotinic acid

2-hydroxynicotinic acid (75 mg, 0.54 mmol) was dissolved in MeOH (0.75 mL) and H₂O (0.112 mL). Grinded KOH (60 mg g, 1.07 mmol) was added and the reaction mixture was refluxed for 15 min. MeI (0.389 mL, 6.03 mmol) was added and the reaction mixture was refluxed for 2 h. After evaporation to half of the volume and addition of 10% HCl (0.075 mL) white crystals of the title compound were obtained by filtration (38 mg).

¹H NMR (400 MHz, D₂O): δ 8.35 (dd, 1H), 7.93 (dd, 1H), 6.64 (t, 1H), 3.59 (s, 3H)

### b) 3-({9-[2-(2-methoxyphenoxy)benzyl]-3,9-diazaspiro[5.5]undec-3-yl}carbonyl)-1-methylpyridin-2(1H)-one

N-methyl-2-hydroxynicotinic acid (1.2 equiv), 3-[2-(2-methoxyphenoxy)benzyl]-3,9-diazaspiro[5.5]undecane (1.0 equiv), HBTU (1.0 equiv) and Et₃N (1.8 equiv) in CH₂Cl₂ (4 mL) were mixed and stirred for 1 h. The reaction mixture was diluted with Na₂CO₃ (aq. sat) (2 mL) and the product was extracted with CH₂Cl₂ and dried. The pure title compound was obtained by preparative HPLC.

¹H NMR (400 MHz, CDCl3): δ 7.68 (m, 1H), 7.56 (m, 1H), 7.42 (m, 1H), 7.27 (m, 3H), 7.06 (m, 4H), 6.61 (m, 1H), 6.29 (m, 1H), 3.73 (m, 3H), 3.54 (m, 3H), 3.29 (m, 1H), 3.03 (m, 1H), 2.33 (m, 4H), 2.08 (m, 1H), 1.85 (m, 1H), 1.62 (m, 1H)

APCI-MS m/z: 501 [MH⁺],

HPLC (Method A) RT: 6.79 min,

HPLC (Method B) RT: 8.08 min

### EXAMPLE 2

### 3-({9-[2-(2-methoxyphenoxy)benzyl]-3,9-diazaspiro[5.5]undec-3-yl}carbonyl)pyridin-2(1H)-one

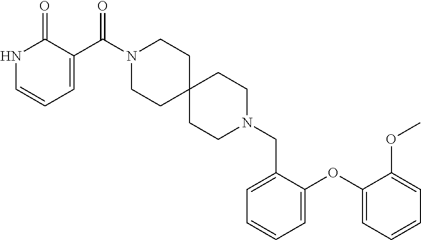

The title compound was synthesized according to Example 1b, but using 2-hydroxynicotinic acid (33 mg, 0.20 mmol) and intermediate C (2 mL (0.1 M), 0.20 mmol). The crude product was purified with preparative HPLC (RP-18, gradient acetonitrile/water/TFA 20/80/0.1 to 50/50/0.1) to afford 15 mg (13%) of the title compound as a slightly yellow solid.

¹HNMR (400 MHz, DMSO-D6) δ 11.86 (s, 1H), 9.11 (s, 1H), 7.57 (d, J=7.5 Hz, 1H), 7.45 (d, J=5.0 Hz, 2H), 7.38-7.26 (m, 2H), 7.25-7.09 (m, 3H), 7.08-7.00 (m, 1H), 6.56-6.48 (m, 1H), 6.23 (t, J=6.5 Hz, 1H), 4.47 (d, J=4.3 Hz, 2H), 3.70 (d, J=5.6 Hz, 3H), 3.58-3.51 (m, 2H), 3.42-3.30 (m, 3H), 3.27-3.07 (m, 4H), 1.99-1.84 (m, 2H), 1.67-1.48 (m, 4H), 1.41-1.29 (m, 2H)

APCI-MS m/z: 488.3 [MH+]

HPLC (Method A) RT: 6.39 min

HPLC (Method B) RT: 7.77 min

### EXAMPLE 3

### 5-({9-[2-(2-methoxyphenoxy)benzyl]-3,9-diazaspiro[5.5]undec-3-yl}carbonyl)-6-(trifluoromethyl)pyridin-2(1H)-one trifluoroacetate

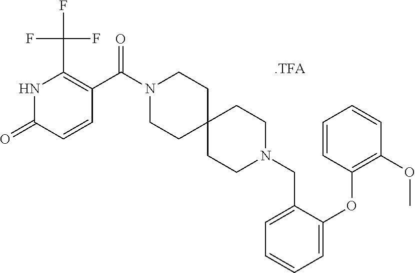

A mixture of intermediate C (88 mg, 0.20 mmol), N-[(dimethylamino)(3H-[1,2,3]triazolo[4,5-b]pyridin-3-yloxy)methylene]-N-methylmethanaminium hexafluorophosphate (HATU, 91 mg, 0.24 mmol), 6-oxo-2-(trifluoromethyl)-1,6-dihydropyridine-3-carboxylic acid (50 mg, 0.24 mmol), triethylamine (200 μl, 1.4 mmol) and dichloromethane (2 mL) was stirred at ambient temperature for 1 h and evaporated. Product was purified twice with preparative HPLC (RP-18, gradient acetonitrile/water/NH4OAc 10/90/0.1 to 95/5/0.1 and acetonitrile/water/TFA 10/90/0.1) to give the product as a white solid (26 mg, 19%).

¹H NMR (400 MHz, CD3OD with NaOD added): δ 7.68 (dd, 1H), 7.51 (t, 1H), 7.40-7.24 (m, 2H), 7.23-7.00 (m, 4H), 6.95 (d, J=1H), 6.60 (t, J=7.2 Hz, 1H), 4.52 (d, 2H), 3.79-3.70 (m, 5H), 3.51 (d, 2H), 3.37-3.19 (m, 2H), 2.04 (t, 2H), 1.88-1.35 (m, 6H)

APCI-MS m/z: 422.5 [MH+]

HPLC (Method A) RT: 7.61 min

HPLC (Method B) RT: 5.31 min

### EXAMPLE 4

### 3-({9-[3-(2-methoxyphenoxy)benzyl]-3,9-diazaspiro[5.5]undec-3-yl}carbonyl)pyridin-2(1H)-one

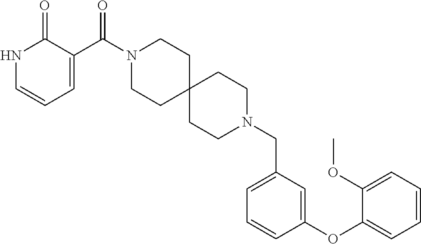

The title compound was synthesized according to Example 3, but using 2-hydroxynicotinic acid (33 mg, 0.20 mmol) and intermediate D (2 mL (0.1 M), 0.20 mmol). The crude product was purified with preparative HPLC (RP-18, gradient acetonitrile/water/TFA 20/80/0.1 to 50/50/0.1) to afford 15 mg (13%) of the title compound as a white solid.

¹HNMR (299.945 MHz, CD3OD) δ 7.66-7.60 (m, 1H), 7.56-7.49 (m, 1H), 7.44-7.36 (m, 1H), 7.24 (t, J=7.7 Hz, 1H), 7.17-6.91 (m, 6H), 6.45 (td, J=6.7, 3.4 Hz, 1H), 4.27 (d, J=6.8 Hz, 2H), 3.76 (s, 3H), 3.74-3.68 (m, 2H), 3.38-3.03 (m, 6H), 2.01 (d, J=13.6 Hz, 2H), 1.82-1.68 (m, 2H), 1.68-1.44 (m, 4H)

APCI-MS m/z: 488.3 [MH+]

HPLC (Method A) RT: 6.25 min

HPLC (Method B) RT: 7.67 min

### Pharmacological Data

### CCL1 SPA Binding Assay

Membranes from CHO-K1 cells transfected with human recombinant chemokine CCR8 receptor (ES-136-M) were purchased from Euroscreen. Membrane preparations are stored at −70 C in 7.5 mM Tris-Cl pH 7.5, 12.5 mM MgCl₂, 0.3 mM EDTA, 1 mM EGTA, 250 mM sucrose until used.

The CCR8 membranes (50.6 mg/ml) were preincubated with Wheat Germ Agglutinin SPA beads (4.05 mg/ml) in assay buffer (50 mM HEPES, 1 mM CaCl₂x2H₂O, 5 mM MgCl₂x6H₂O, 75 mM NaCl, 0.1% BSA) at pH=7.4 for 2 hours on ice. A 10-point dose-response curve (final concentrations 50 μM, 16.7 μM, 5.6 μM, 1.9 μM, 0.62 μM, 0.21 μM, 0.069 μM, 0.023 μM) was prepared by diluting compounds by serial dilution 1:3 in DMSO. In the screening plate (Polystyrene NBS plates, Costar Corning 3604) 1 μl from the DMSO solutions of compounds was transferred into each well. 1 μl of DMSO was added to the blank control wells and 1 μl unlabeled CCL1 (300 nM) was added to background control wells. 50 μl of the SPA bead-membrane mixture was added into each well. Finally, 50 μl. (30 μM) ¹²⁵I CCL1 (2000 Ci/mM) was added to each well. Plates were then incubated at RT with shaking (700 rpm) for 90 minutes followed by 30 minutes at RT without shaking. The plate was read in a Wallac MicroBeta counter for 2 minutes/well.

### Human Microsomal Stability Assay

The assay is run in a 96-deepwell format at 1 mg microsomal protein (Xenotech)/mL in potassium phosphate buffer (pH 7.4) with a compound concentration of 2.5 μM and a NADPH concentration of 2 mM. Samples at four time-points (0, 5, 15 and 30 minutes) are withdrawn and the enzymatic reaction is terminated by protein precipitation with 1% acetic acid in acetonitrile. The incubations are performed on a thermostated plate (37° C.) placed on a Tecan worktable, and all liquid handling was performed robotically. After centrifugation of the samples, the supernatants are pooled in sets of four before they are analysed by liquid chromatography with tandem mass spectrometry detection (LC/MS/MS) using multiple reaction monitoring (MRM). Data are presented as intrinsic Clearance (CLᵢₙₜ), μl/min/mg protein, calculated from the initial linear part of the compound disappearance curve.

Tables 1 and 2 show the results that were obtained when the compounds of Examples 1 to 4 above were tested in the above-described CCL1 SPA binding assay (expressed as 1050 values) and human microsomal stability assay. Data is also shown for four comparison compounds (A, B, X and Y).

Comparative examples A, B, X and Y are the following:

### A: 3-{[9-(2-isobutoxybenzyl)-3,9-diazaspiro[5.5]undec-3-yl]carbonyl}pyridin-2(1H)-one

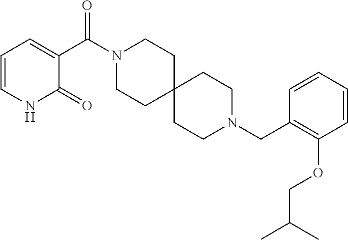

### B: 5-{[9-(2-isobutoxybenzyl)-3,9-diazaspiro[5.5]undec-3-yl]carbonyl}pyridin-2(1H)-one

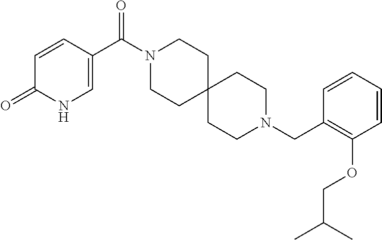

### X: 3-isonicotinoyl-9-[2-(2-methoxyphenoxy)benzyl]-3,9-diazaspiro[5.5]undecane

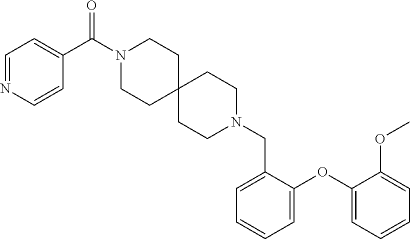

### Y: 3-isonicotinoyl-9-[3-(2-methoxyphenoxy)benzyl]-3,9-diazaspiro[5.5]undecane

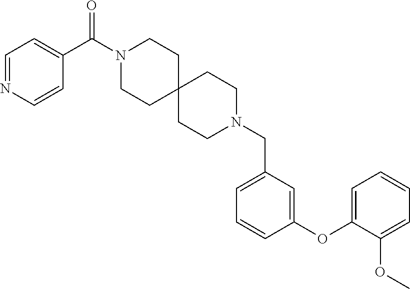

TABLE 1

| Example No. | CCR8 binding (μM) |
|---|---|
| A | 1.950 |
| B | 1.900 |
| 1 | 0.044 |
| 2 | 0.062 |
| 3 | 0.083 |
| 4 | 0.015 |

TABLE 2

| Example No. | Human Microsomal Stability μl/min/mg) |
|---|---|
| 1 | 15.7 |
| 2 | <10 |
| 3 | <10 |
| 4 | 21.7 |
| X | 89 |
| Y | 183 |

1. A compound of formula (II): 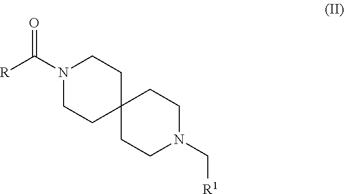
wherein
R is pyridin-2-one or N—C₁-C₄ alkyl pyridin-2-one; or R is pyridin-2-one or N—C₁-C₄ alkyl pyridin-2-one each of which is substituted with a group selected from CF₃, halogen or C₁-C₄ alkyl;
R¹ represents the group: 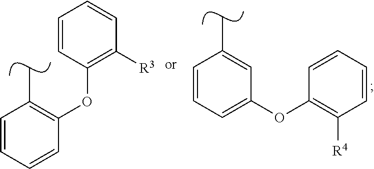
and R³ and R⁴ are independently methoxy or ethoxy;
or a pharmaceutically acceptable salt thereof.

2. The compound according to claim 1, wherein R³ and R⁴ are methoxy.

3. The compound according to claim 1, wherein said N—C₁-C₄ alkyl pyridin-2-one is N-methylpyridin-2-one.

4. The compound according to claim 1, wherein R is pyridin-2-one or N—C₁-C₄ alkyl pyridin-2-one or R is pyridin-2-one or N—C₁-C₄ alkyl pyridin-2-one substituted with a single CF₃ group.

5. The compound according to claim 1 selected from the following or a pharmaceutically acceptable salt thereof:
3-({9-[2-(2-methoxyphenoxy)benzyl]-3,9-diazaspiro[5.5]undec-3-yl}carbonyl)-1-methylpyridin-2(1H)-one,
3-({9-[2-(2-methoxyphenoxy)benzyl]-3,9-diazaspiro[5.5]undec-3-yl}carbonyl)pyridin-2(1H)-one,
5-({9-[2-(2-methoxyphenoxy)benzyl]-3,9-diazaspiro[5.5]undec-3-yl}carbonyl)-6-(trifluoromethyl)pyridin-2(1H)-one, and
3-({9-[3-(2-methoxyphenoxy)benzyl]-3,9-diazaspiro[5.5]undec-3-yl}carbonyl)pyridin-2(1H)-one.

6. A pharmaceutical composition comprising the compound according to claim 1 or a pharmaceutically acceptable salt thereof, and a pharmaceutically acceptable adjuvant, diluent or carrier.

7-9. (canceled)

10. A method of treating as which comprises administering to a patient in need thereof a therapeutically effective amount of the compound according to claim 1, or a pharmaceutically-acceptable salt thereof.

11. A method of treating chronic obstructive pulmonary disease which comprises administering to a patient in need thereof a therapeutically effective amount of the compound according to claim 1, or a pharmaceutically-acceptable salt thereof.

12. A method of treating rhinitis which comprises administering to a patient in need thereof a therapeutically effective amount of the compound according to claim 1, or a pharmaceutically-acceptable salt thereof.

13. (canceled)

14. A method of treating a chemokine mediated disease wherein the chemokine binds to one or more chemokine receptors, which comprises administering to a patient in need thereof a therapeutically effective amount of the compound according to claim 1, or a pharmaceutically-acceptable salt thereof.

15. The method according to claim 14 in which the chemokine receptor is the CCR8 receptor.

16. A method of treating a respiratory disease, which comprises administering to a patient in need thereof a therapeutically effective amount of the compound according to claim 1, or a pharmaceutically acceptable salt thereof.

17. The method according to claim 16, wherein the respiratory disease is selected from the group consisting of asthma and chronic obstructive pulmonary disease.

18. A process for the preparation of the compound according to claim 1 which comprises
(a) reacting a compound of formula (III): 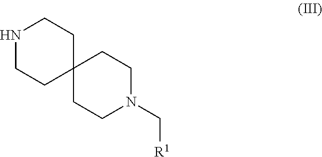
where R¹ is as defined in claim 1, with a compound of formula (IV): 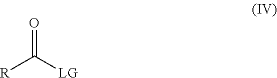
where R is as defined in claim 1 and LG is a suitable leaving group, or
(b) reacting a compound of formula (V) 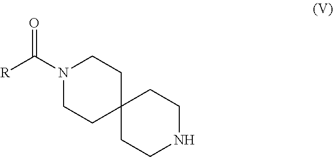
wherein R is as defined in claim 1, with an aldehyde compound of formula (VI): 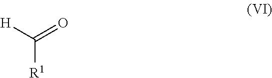
wherein R¹ is as defined in claim 1, or
(c) reacting a compound of formula (V) as defined in (b) with a compound of formula (VII) 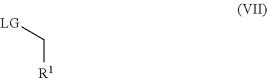
wherein R¹ is as defined in claim 1 and LG is a leaving group.
